(19) United States
(12) Patent Application Publication
(10) Pub. No.: US 2026/0152693 A1
(43) Pub. Date: Jun. 4, 2026

(54) PYROLYSIS APPARATUS, METHOD, AND SYSTEM FOR LOSS RECOVERY AND CORRECTION OF LIGHT HYDROCARBONS IN MUD SHALE

(71) Applicants: CHINA PETROLEUM & CHEMICAL CORPORATION, Beijing (CN); SINOPEC EXPLORATION & PRODUCTION RESEARCH INSTITUTE, Beijing (CN)

(72) Inventors: Menhui QIAN, Beijing (CN); Changbo ZHAI, Beijing (CN); Maowen LI, Beijing (CN); Zhiming LI, Beijing (CN); Baojian SHEN, Beijing (CN); Yunying LENG, Beijing (CN)

(21) Appl. No.: 18/995,824

(22) PCT Filed: Feb. 6, 2023
(86) PCT No.: PCT/CN2023/074562

§ 371 Date: Jan. 16, 2025

(30) Foreign Application Priority Data
Jul. 19, 2022 (CN) 202210851318.5

(51) Int. Cl. C10G 1/02; B01J 6/00; C07C 4/04

(52) U.S. Cl. CPC C10G 1/02; B01J 6/008; C07C 4/04; C10G 2300/1033

(57) ABSTRACT

A pyrolysis apparatus, method, and system for loss recovery and correction of light hydrocarbons in mud shale are provided. The pyrolysis apparatus has a pyrolysis tank for accommodating and heating a full-diameter core sample, an input pipeline and an output pipeline being respectively arranged at two opposite ends of the pyrolysis tank, and a flow divider assembly being arranged on the output pipeline, forming a first branch pipeline and a second branch pipeline; a carrier gas assembly in communication with the input pipeline; and a detection assembly, comprising a first detection unit for quantitative detection and a second detection unit for qualitative detection, which are connected to the first branch pipeline and the second branch pipeline, respectively. The pyrolysis apparatus, method, and system for loss recovery and correction of light hydrocarbons in mud shale carry out on-site pyrolysis of full-diameter core.

### TECHNICAL FIELD

The present invention relates to the technical field of oil/gas exploration, and specifically to a pyrolysis apparatus, method, and system for loss recovery and correction of light hydrocarbons in mud shale.

### TECHNICAL BACKGROUND

In the oil/gas exploration of unconventional shale, researchers pay particular attention to light hydrocarbons due to low carbon number and excellent fluidity thereof. The amount and components of light hydrocarbons are among the key parameters in the exploration and development of oil and gas in shale. The light hydrocarbons in mud shale core generally have carbon numbers below C15, which are extremely volatile. At present, the most direct and objective data are obtained generally through on-site experiments on block core or full-diameter core, and the combined quantitative and qualitative detection of light hydrocarbons using block core is the main research direction. Existing methods and devices can be generally divided into three types as follows.

The first type relates to realizing quantitative and qualitative analysis of hydrocarbons by a conventional rock pyrolysis instrument and a chromatographic component analyzer externally connected thereto. However, such method is only applicable to powder samples due to the limitation of feeding system, while studies have found that preparation of powdery sample will lead to serious loss of light hydrocarbons.

The second type relates to a specially designed sealed crushing tank, for crushing block samples in a sealed state. Since the volume of light hydrocarbons expands after crushing, the pressure in the sealed crushing tank will increase. Then, quantitative conversion based on pressure changes or quantitative measurement through drainage method is performed, followed by qualitative analysis with online chromatography. However, such method actually measures gaseous hydrocarbon (C1-C4) in the light hydrocarbons, but is not applicable to liquid hydrocarbons (C5+) with smaller coefficient of compressibility, which may result in large error of quantitative result. Direct thermal release performed on block core or full-diameter core is often applied to the analysis of shale gas content. However, such device has no carrier gas system, and can only achieve “pressurization” by heating core sample. Therefore, it is only applicable to the determination of gaseous hydrocarbon content, failing to meet the requirements for the determination of light hydrocarbon content in mud shale.

The third type carries out sealed crushing and extraction through solvents with low boiling point, which requires rectification and purification of solvents such as Freon. Meanwhile, such experiment has complex procedures, and needs to be carried out at a low temperature (usually below 18° C.), with strict requirements for the environment and experimental technology. Moreover, it can only measure light hydrocarbon components of C6-C15, instead of all light hydrocarbon contents.

In addition, part of the light hydrocarbons in the core sample will be volatilized and lost during collection, extraction and subsequent storage procedures, which will lead to errors in the data obtained from subsequent pyrolysis of the core sample and also in various parameters of the core determined therefrom. During pyrolysis analysis of the core sample, it is therefore necessary to determine the amount of lost light hydrocarbons during the collection and extraction procedures of the core sample first, in order to obtain the final various parameters of the core accurately. However, existing means for determining the loss of volatilized light hydrocarbons all have defects to varying degrees.

Therefore, it is necessary to design a pyrolysis apparatus, method, and system for loss recovery and correction of light hydrocarbons in mud shale, in order to overcome the defects in the existing methods as mentioned above.

### SUMMARY OF THE INVENTION

In view of the above technical problems, the present invention proposes a pyrolysis apparatus, method, and system for loss recovery and correction of light hydrocarbons in mud shale.

According to a first aspect of the present invention, a pyrolysis apparatus for loss recovery and correction of light hydrocarbons in mud shale is proposed, which comprises:
a pyrolysis tank, for accommodating and heating a full-diameter core sample, an input pipeline and an output pipeline being respectively arranged at two opposite ends of the pyrolysis tank, and a flow divider assembly being arranged on the output pipeline, forming a first branch pipeline and a second branch pipeline;
a carrier gas assembly in communication with the input pipeline; and
a detection assembly, comprising a first detection unit for quantitative detection and a second detection unit for qualitative detection, which are connected to the first branch pipeline and the second branch pipeline, respectively,
wherein the pyrolysis tank comprises an oval columnar tank body, a heating mechanism is arranged on a side wall of the tank body, and a vertical air grille structure is arranged on each of front and rear sides of the tank body, the vertical air grille structure including several gas holes vertically arranged in a row.

In one embodiment, the first detection unit includes a hydrogen flame ionization detector connected to a combustion mechanism, and the second detection unit includes a chromatographic detector.

In one embodiment, the flow divider assembly comprises a triple valve and a flow divider valve, wherein the triple valve is configured to separate the output pipeline to form the first branch pipeline and the second branch pipeline, and the flow divider valve is arranged on the first branch pipeline or the second branch pipeline, for controlling a flow distribution proportion of the first branch pipeline to the second branch pipeline, the flow divider valve being configured as a high-temperature resistant metal flow divider valve.

In one embodiment, the apparatus further comprises a thermal insulation box arranged at an output end of the pyrolysis tank, wherein the output pipeline is arranged inside the thermal insulation box.

In one embodiment, a third branch pipeline is formed at an end of the second branch pipeline and configured as an exhaust pipeline with an exhaust valve, and
the first branch pipeline and the second branch pipeline are in a normally open state under a normal pressure.

In one embodiment, temperature sensors are arranged on the pyrolysis tank at least in a position where the heating mechanism is located and at an output end thereof corresponding to the output pipeline, and
the tank body is of a multi-layer structure, the heating mechanism is configured as an annular heating sheet arranged as a middle layer of the tank body, and a quick-release structure is arranged on the tank body and comprises a cover arranged on a top portion of the tank body and connected to a core basket through a vertical connecting member.

According to a second aspect of the present invention, a loss recovery and correction method for light hydrocarbons in mud shale is proposed, comprising:
determining, based on hydrocarbon signal in response to hydrocarbons in a pyrolysis product of a core sample, a relationship between an accumulated amount of pyrolyzed light hydrocarbons of the core sample and a pyrolysis time;
determining, according to said relationship, an accumulated amount of light hydrocarbon loss of the core sample during a time period after extraction from target formation and prior to pyrolysis; and
determining, according to the accumulated amount of light hydrocarbon loss, a light hydrocarbon correction coefficient.

In one embodiment, the hydrocarbon signal of the hydrocarbons in the pyrolysis product of the core sample is detected by said pyrolysis apparatus.

In one embodiment, determining, according to said relationship, the accumulated amount of light hydrocarbon loss of the core sample during the time period after extraction from target formation and prior to pyrolysis comprises:
determining, according to the hydrocarbon signal, a scatter diagram of the accumulated amount of pyrolyzed light hydrocarbons and the pyrolysis time within a time period after the pyrolysis starts;
fitting a target curve representing said relationship according to the scatter diagram; and
regressing the target curve to an initial time point when the pyrolysis starts, and taking the accumulated amount of pyrolyzed light hydrocarbons corresponding to the initial time point as the accumulated amount of light hydrocarbon loss.

In one embodiment, determining, according to the accumulated amount of light hydrocarbon loss, the light hydrocarbon correction coefficient comprises:
determining the light hydrocarbon correction coefficient according to the accumulated amount of light hydrocarbon loss, a total accumulated amount of pyrolyzed light hydrocarbons during the pyrolysis of the core sample, and synchronously-measured free oil content in the core sample.

In one embodiment, the light hydrocarbon correction coefficient is determined with an expression as follows:

$$w = ( {S}_{1} + {C}_{1} ) / ( {S}_{1} - {C}_{2} ) ,$$

wherein S₁ denotes the free oil content in the core sample, C₁ denotes the accumulated amount of light hydrocarbon loss, and C₂ denotes the total accumulated amount of pyrolyzed light hydrocarbons.

In one embodiment, the free oil content is measured through steps of:
separating and freezing a sub-sample from the core sample before the pyrolysis of the core sample; and
pyrolyzing the frozen sub-sample and determining the free oil content based on an amount of pyrolysis product of the sub-sample.

In one embodiment, the total accumulated amount of pyrolyzed light hydrocarbons is measured through steps of:
determining a total accumulated amount of hydrocarbon signal when the hydrocarbon signal stops; and
determining the total accumulated amount of pyrolyzed light hydrocarbons of the core sample according to the total accumulated amount of hydrocarbon signal.

In one embodiment, determining the total accumulated amount of pyrolyzed light hydrocarbons of the core sample according to the total accumulated amount of hydrocarbon signal comprises:
generating a calibration curve based on a response to the hydrocarbon signal of the pyrolysis of a standard light oil sample; and
determining the total accumulated amount of pyrolyzed light hydrocarbons of the core sample based on the calibration curve and the total accumulated amount of hydrocarbon signal.

In one embodiment, the method further comprises:
collecting the pyrolysis product of the core sample in different time periods, in order to determine the amount of pyrolyzed hydrocarbons, proportions of hydrocarbon components and the content of each component corresponding to said different time periods.

According to a third aspect of the present invention, a pyrolysis system for loss recovery and correction of light hydrocarbons in mud shale is proposed, comprising said pyrolysis apparatus, and a data processing apparatus electrically connected thereto.

The above technical features may be combined in various suitable manners or replaced with equivalents thereof, as long as the purpose of the present invention can be achieved.

Compared with the prior arts, the pyrolysis apparatus, method, and system for loss recovery and correction of light hydrocarbons in mud shale according to the present invention may have at least the following beneficial effects.

The pyrolysis apparatus, method, and system for loss recovery and correction of light hydrocarbons in mud shale according to the present invention provides a technical solution that can carry out on-site pyrolysis of full-diameter core, which effectively avoids light hydrocarbon loss caused by rock fragmentation, and realizes quantitative and qualitative pyrolysis of light hydrocarbons through flow distribution of high-temperature gas. Moreover, the technical solution according to the present invention can determine the relationship between the accumulated amount of pyrolyzed light hydrocarbons and the pyrolysis time, and further obtains the light hydrocarbon recovery correction coefficient by calculating the amount of light hydrocarbon loss in the core during tripping and the total amount of pyrolyzed light hydrocarbons in the core on site. On this basis, the actual oil content of corresponding formations and lithofacies can be obtained, so that the shale oil resource can be evaluated accurately.

### BRIEF DESCRIPTION OF THE DRAWINGS

The present invention will be described below in more detail based on the embodiments with reference to the accompanying drawings.

FIG. 1 schematically shows a structure of a pyrolysis apparatus according to the present invention.

FIG. 2 schematically shows a structure of a pyrolysis tank according to the present invention.

FIG. 3 is a flow diagram of the method according to the present invention.

FIG. 4 shows a calibration curve in the method according to the present invention.

FIG. 5 is a diagram showing pyrolysis data in one embodiment according to the present invention.

FIG. 6 shows a curve indicating a relationship between an accumulated amount of pyrolyzed light hydrocarbons and pyrolysis time obtained based on the pyrolysis data in FIG. 5.

In the drawings, the same reference signs are used to indicate the same components. The drawings are not necessarily drawn to actual scales.

### DETAILED DESCRIPTION OF EMBODIMENTS

The volatilization of light hydrocarbon compound is subject to van der Waals forces. For light hydrocarbon compounds with similar molecular structures, the higher the molecular weight, the lower the volatile loss. For compounds with the same molecular weight, the higher the boiling point, the lower the volatile loss. Concerning light hydrocarbon molecules with different structures, branched and linear alkanes are more volatile than cycloalkanes. Studies have shown that there are losses of light hydrocarbon components in the collection, transportation, storage and pre-treatment of typical samples in shale formations. Therefore, in the geological evaluation of shale oil, including oil bearing property, mobility and resource potential evaluation of shale, the correction (recovery) of light hydrocarbon loss has become a focus of attention for many scholars.

There are few existing studies on the recovery technology of volatile loss of light hydrocarbons (C1-C15), and different points of view are held. Some scholars believe that most of light hydrocarbons are lost during sample storage and processing procedures, accounting for about 35% (C14−/C5+), while others believe that crude oil contains 30% light hydrocarbons. Still others hold that about half of the residual hydrocarbons are lost during core standing as well as analysis and testing procedures.

At present, the methods for the recovery of light hydrocarbon volatile loss can be generally divided into the following types. The first method is the recovery technology of light hydrocarbon volatile loss based on chemical kinetic parameters and mass balance, and the second method is to estimate the loss of C15-light hydrocarbons based on density index API of crude oil and the hydrocarbon content below C15. In these two methods, the recovery of light hydrocarbon loss is calculated according to different proportions of light hydrocarbons in source rock due to different thermal evolution degrees of source rock, based on the premise that the light hydrocarbons in source rock are “complete” without migration loss. However, this premise is obviously inconsistent with actual geological situation. The third method is to recover the light hydrocarbon loss during collection of pyrolysis samples based the volume factor of petroleum formation. This method is empirical, because the loss and components of light hydrocarbons during sample storage and preparation are related to various factors, such as the type of sample (rock debris or cores), oil and gas components and contents, maturity of source rock, total organic carbon content, the state of sample (intact or broken), storage conditions after sampling, etc. Thus, this method is not a representative one. The fourth method is to generate a recovery coefficient for light hydrocarbon volatile loss by closed low-temperature extraction using solvent with low boiling point comparing with conventional chloroform, which is a laboratory analysis method that does not take into account the light hydrocarbon loss during sample preparation, transportation and storage. Therefore, the recovery coefficient is inherently defective.

In fact, the loss of light hydrocarbons is a continuous procedure. With different placement time of core samples, there are discrepancies between the content of free hydrocarbons measured in the laboratory. Therefore, the recovery result of light hydrocarbon loss based on measured value after sample placement cannot reflect original oil content of the samples. Additionally, the shale oil system has different openness and migration-and-accumulation characteristics. The sweet spot section of shale oil is generally formed through short-distance migration, while the migration of light hydrocarbons is significantly different from those of medium and heavy hydrocarbons, resulting in different hydrocarbon components in the “source” and “storage” of shale oil. Therefore, the recovery of light hydrocarbon loss based on thermal simulation for hydrocarbon generation may not be well applicable to open or semi-open shale oil systems

The present invention will be described below in more detail with reference to the accompanying drawings.

### Embodiment 1

According to the embodiment of the present invention, a pyrolysis apparatus for loss recovery and correction of light hydrocarbons in mud shale is proposed, which comprises: a pyrolysis tank 2, for accommodating and heating a full-diameter core sample 8, wherein an input pipeline 21 and an output pipeline 22 are respectively arranged at two opposite ends of the pyrolysis tank 2, and a flow divider assembly 3 is arranged on the output pipeline 22, forming a first branch pipeline 221 and a second branch pipeline 222; a carrier gas assembly 1 in communication with the input pipeline 21; and a detection assembly 4, comprising a first detection unit 41 for quantitative detection and a second detection unit 42 for qualitative detection, which are connected to the first branch pipeline 221 and the second branch pipeline 222, respectively, wherein the first detection unit 41 is a quantitative detector, preferably a hydrogen flame ionization detector, and the second detection unit 42 is a qualitative detector, preferably a chromatographic detector.

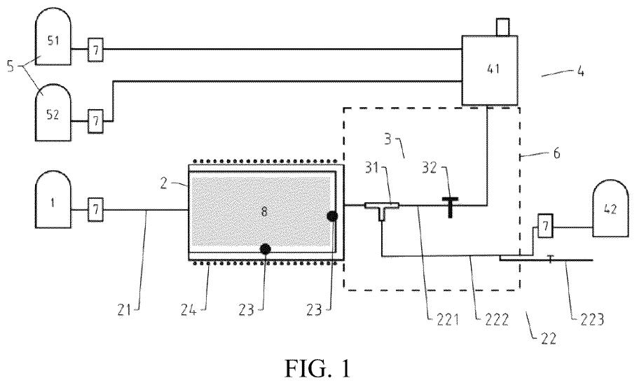

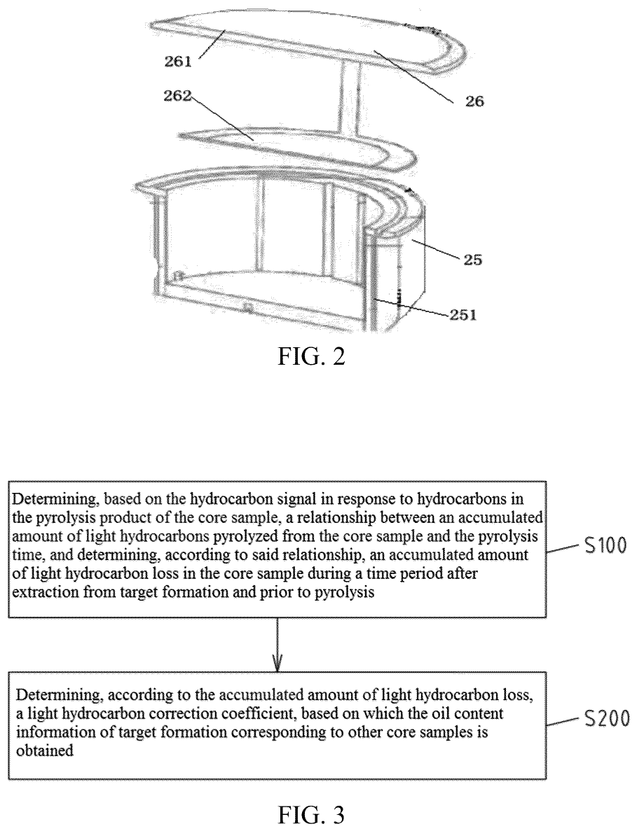

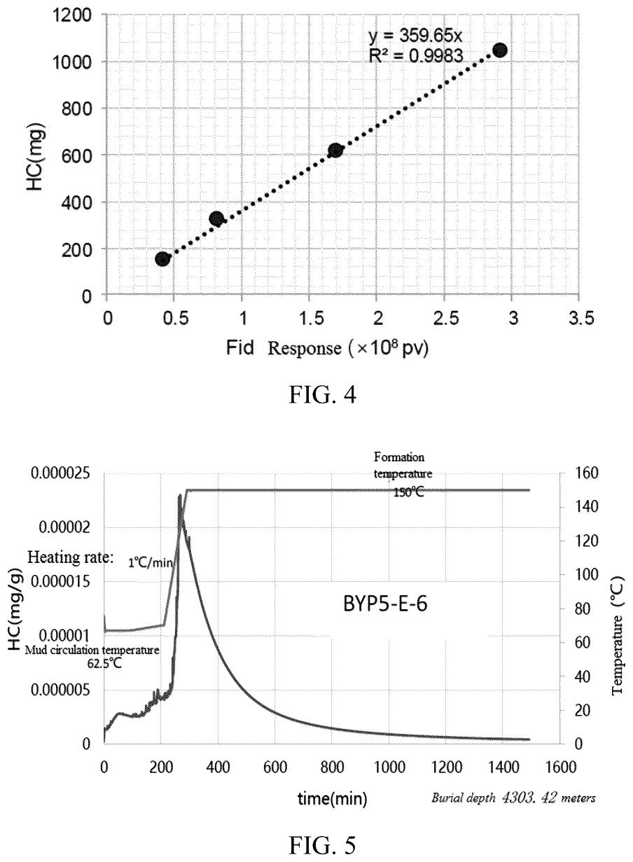

Specifically, as shown in FIG. 1, the pyrolysis apparatus according to the present invention mainly comprises three parts, namely the pyrolysis tank 2, the carrier gas assembly 1 and the detection assembly 4. The pyrolysis tank 2, which is configured to heat and pyrolyze the core sample 8, comprises the input pipeline 21 for carrier gas input and the output pipeline 22 for product output at both ends thereof. The carrier gas assembly 1 is configured to input carrier gas into the pyrolysis tank 2. The carrier gas functions to drive pyrolysis product of the core sample 8 to flow and provide power for the output thereof. The detection assembly 4, which is configured to detect the pyrolysis product of the core sample 8, includes two detection units, namely the first detection unit 41 for quantitative detection and the second detection unit 42 for qualitative detection. The first detection unit 41 and the second detection unit 42 are respectively connected to the first branch pipeline 221 and the second branch pipeline 222 formed at a rear end of the output pipeline 22 of the pyrolysis tank 2, so as to realize quantitative and qualitative detections of the pyrolysis product of the core sample 8.

The pyrolysis tank 2 comprises an oval columnar tank body. A quick-release structure is arranged at an upper portion of the tank body, and a core basket is arranged below the quick-release structure. A heating mechanism and a vertical air grille structure are arranged on the tank body. The oval tank body is suitable for the measurement of full-diameter core, thus reducing the hydrocarbon residual coefficient. The core basket and the quick-release structure facilitate the extraction of the core.

In operation, the core sample 8 is placed in the pyrolysis tank 2 and heated according to a preset heating scheme, wherein the heating is maintained during the pyrolysis. At the same time, the carrier gas assembly 1 is activated to deliver the carrier gas to the pyrolysis tank 2. The pyrolysis product is output driven by the carrier gas and mixed evenly with the carrier gas. The pyrolysis product enters the first branch pipeline 221 and the second branch pipeline 222 respectively via the output pipeline 22. The pyrolysis product entering the first branch pipeline 221 and that entering the second branch pipeline 222 reach the first detection unit 41 and the second detection unit 42 for respective detections.

Further, the first detection unit 41 includes a hydrogen flame ionization detector connected to a combustion mechanism 5, and the second detection unit 42 includes a chromatographic detector.

Specifically, referring to FIG. 1, in this embodiment the hydrogen flame ionization detector is used as the first detection unit 41 for quantitative detection, and the chromatographic detector is used as the second detection unit 42 for qualitative detection. The pyrolysis product of the core sample 8 reaches the hydrogen flame ionization detector and the chromatographic detector through the first branch pipeline 221 and the second branch pipeline 222, respectively.

The hydrogen flame ionization detector can detect hydrocarbon components in the pyrolysis product and collect hydrocarbon signal. According to a predetermined relationship between an accumulated amount of hydrocarbon signal of the sample to be tested by the hydrogen flame ionization detector and a mass of the sample to be tested, the amount of hydrocarbons reaching the hydrogen flame ionization detector can be determined. Then, according to a flow distribution proportion of the first branch pipeline 221 to the second branch pipeline 222, a total amount of hydrocarbons output by the output pipeline 22 can be determined. According to a proportion of the total amount of hydrocarbons to a mass of the core sample 8, a total hydrocarbon content of the core sample 8 is determined, thus achieving the quantitative detection. The hydrogen flame ionization detector is connected to the combustion mechanism 5, which includes a combustion gas unit 51 (hydrogen) and a combustion-supporting gas unit 52 (oxygen).

The chromatographic detector can detect, through chromatographic analysis, the amount of pyrolyzed hydrocarbons in the pyrolysis product, and the proportions of different components in the hydrocarbons and the content of each component. Meanwhile, since the chromatographic detector detects on a regular basis, the amount of pyrolyzed hydrocarbons as well as the proportions of hydrocarbon components and the content of each component in different pyrolysis period can be further calculated according to time intervals between collections, in order to perform qualitative and quantitative analysis of hydrocarbons in different volatilization stages.

Moreover, the apparatus further includes a thermal insulation box 6 arranged at an output end of the pyrolysis tank 2, and the output pipeline 22 is arranged inside the thermal insulation box 6.

Specifically, as shown in FIG. 1, the output pipeline 22 is basically arranged inside the thermal insulation box 6, except for a connecting portion at an end thereof to be connected to the corresponding detection unit. The thermal insulation box 6 is mainly used for thermal insulation of the output pipeline 22, in order to ensure the normal flow of gas-phase pyrolysis product and avoid the reduction in temperature that would affect the normal flow of the pyrolysis product and even lead to the condensation of the pyrolysis product.

Further, the flow divider assembly 3 includes a triple valve 31 and a flow divider valve 32. The triple valve 31 separates the output pipeline 22 to form the first branch pipeline 221 and the second branch pipeline 222, and the flow divider valve 32 is arranged on the first branch pipeline 221 or the second branch pipeline 222.

Specifically, as shown in FIG. 1, the flow divider assembly 3 includes the triple valve 31 and the flow divider valve 32, wherein the triple valve 31 is configured to separate the output pipeline 22 to form the first branch pipeline 221 and the second branch pipeline 222. The flow divider valve 32, which is high-temperature resistant and mainly configured to adjust and control flow rate, is arranged on one of the branch pipelines to control a flow rate thereof. In this case, when a total flow rate is controlled at a certain value, a flow rate at the other branch pipeline is thus controlled, in order to achieve proportional distribution of flow.

Further, a third branch pipeline 223, which is an exhaust pipeline with an exhaust valve, is formed at an end of the second branch pipeline 222.

Specifically, as shown in FIG. 1, the third branch pipeline 223 is configured to exhaust the product therein. For example, before the hydrocarbon pyrolysis product reaches the second detection unit 42 arranged at the end of the second branch pipeline 222, the second detection unit 42 is in a closed state to avoid introducing other impurities at a front end of the hydrocarbon pyrolysis product. At this time, the exhaust valve can be activated to turn on the third branch pipeline 223 for exhausting the impurities.

Further, temperature sensors 23 are arranged on the pyrolysis tank 2, at least in a position where a heating unit 24 thereof is located and at an output end thereof corresponding to the output pipeline 22.

Specifically, as shown in FIG. 1, the temperature sensors 23 are configured to detect a temperature of the pyrolysis tank 2, in order to ensure the accuracy of the heating temperature and thus achieve the predetermined heating scheme. The temperature sensors 23 are arranged in different positions of the pyrolysis tank 2, specifically at least in the position where the heating unit 24 of the pyrolysis tank 2 is located (on a circumferential wall of the pyrolysis tank 2) and at the output end of the pyrolysis tank 2 corresponding to the output pipeline 22 (on an end surface of the pyrolysis tank 2), as shown in FIG. 1. Such arrangements can not only ensure the accuracy of the heating temperature but also meet the requirement for the temperature of pyrolysis product output.

Further, as shown in FIG. 1, in this embodiment flow controllers 7 are provided at an output end of the carrier gas assembly 1, an output end of the combustion mechanism 5, and an input end of the second detection unit 42, for controlling material flow at corresponding structures.

According to a proportion of a total flow rate detected by the flow controller 7 at the output end of the carrier gas assembly 1 to a sub-flow rate at the second detection unit 42 detected by the flow controller 7 at the input end thereof, a flow rate at the first detection unit 41 can be calculated, thus obtaining a flow distribution proportion of the first detection unit 41 to the second detection unit 42.

### Embodiment 2

The embodiment of present invention proposes a loss recovery and correction method for light hydrocarbons in mud shale, and illustrates the principle thereof. The method according to the embodiment comprises:

Step S100: determining, based on the hydrocarbon signal in response to the hydrocarbons in the pyrolysis product of the core sample, a relationship between an accumulated amount of light hydrocarbons pyrolyzed from the core sample and the pyrolysis time, and determining, according to said relationship, an accumulated amount of light hydrocarbon loss in the core sample during a time period after extraction from target formation and prior to pyrolysis.

Specifically, Step 100 may include the following steps as follows:
Step S110: determining, according to the hydrocarbon signal, a scatter diagram of the accumulated amount of pyrolyzed light hydrocarbons and the pyrolysis time within a time period after the pyrolysis starts;
Step S120: fitting a target curve representing said relationship according to the scatter diagram; and
Step S130: regressing the target curve to an initial time point when the pyrolysis starts, and taking the accumulated amount of pyrolyzed light hydrocarbons corresponding to the initial time point as the accumulated amount of light hydrocarbon loss.

Specifically, the hydrocarbons generated by the pyrolysis of the core sample are collected, and the hydrocarbon signal is generated in response thereto. The accumulated amount of pyrolyzed light hydrocarbons during one of different time periods is determined according to a corresponding accumulated amount of hydrocarbon signal during said one time period. Then, the scatter diagram of the accumulated amount of pyrolyzed light hydrocarbons and the pyrolysis time is obtained based on data within a certain period of time after the start of pyrolysis (e.g., 2-4 h). The target curve is fit based on the scatter diagram, and after regressing, an ordinate value of the curve corresponding to the initial time point at the start of pyrolysis (representing the accumulated amount of pyrolyzed light hydrocarbons) is the accumulated amount of light hydrocarbon loss of the core sample during the time period after extraction from target formation and prior to pyrolysis.

The accumulated amount of light hydrocarbon loss is determined in Step S100 (Step S110 to Step S130) based on the principle as follows. Light hydrocarbons in the core sample will continuously volatilize owing to changes in formation temperature and pressure during the extraction of the core sample from target formation (i.e., the procedure of lifting the drill to the ground), resulting in the loss of part of the light hydrocarbons. The principle of such loss is similar to that of the subsequent pyrolysis procedure. Therefore, the amount of such loss can be obtained inversely according to subsequent pyrolysis data. According to a prior analysis of the actual exploration data, it is reasonable to take a median time point during a total time period of drill lifting (from the start of lifting to reaching the wellhead) as a starting time point of the light hydrocarbon loss of the core sample. Therefore, the accumulated amount of light hydrocarbon loss refers to a total volatile loss of hydrocarbons in the core sample from the median time point during the total time period of drill lifting to a time point when the pyrolysis of the core sample starts (including a time period from the wellhead to the pyrolysis apparatus).

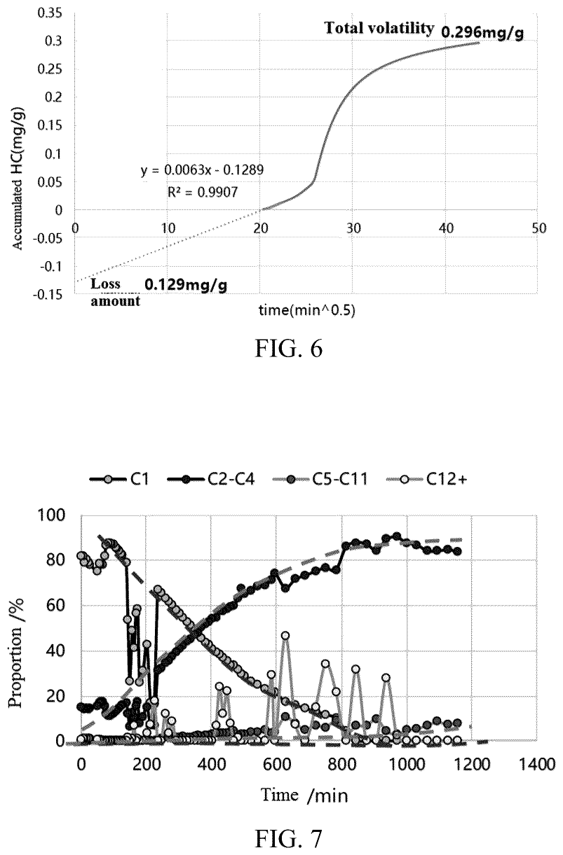

It should be noted that an entire procedure from the starting time point of the light hydrocarbon loss of the core sample (i.e., the median time point during the total time period of drill lifting) to the completion of the pyrolysis can be regarded as a continuous pyrolysis procedure. The first half of the procedure refers to the volatile loss of the core sample from the underground to the ground wellhead and from the ground wellhead to the pyrolysis apparatus, and the second half thereof refers to the pyrolysis in the pyrolysis apparatus. The measurement of the amount of pyrolyzed light hydrocarbons starts from the pyrolysis in the pyrolysis apparatus, and the measured data thereof accumulate from zero (blank baseline). For the convenience of data processing, a relationship between the accumulated amount of pyrolyzed light hydrocarbons and a square root of the pyrolysis time denotes the amount of light hydrocarbon loss in the core sample in an early stage. Therefore, the scatter diagram of the accumulated amount of pyrolyzed light hydrocarbons and the pyrolysis time is one of the accumulated amount of pyrolyzed light hydrocarbons and the square root of the pyrolysis time (t^{0.5}), as shown in FIG. 6.

The method according to the present embodiment further includes:

Step S200: determining, according to the accumulated amount of light hydrocarbon loss, a light hydrocarbon correction coefficient, based on which the oil content information of target formation corresponding to other core samples is obtained.

Specifically, Step 200 may include:

Step S210: determining the light hydrocarbon correction coefficient according to the accumulated amount of light hydrocarbon loss, the total accumulated amount of pyrolyzed light hydrocarbons during the pyrolysis of the core sample, and synchronously-measured free oil content in the core sample. The light hydrocarbon correction coefficient can be determined with an expression as follows:

$$w = ( {S}_{1} + {C}_{1} ) / ( {S}_{1} - {C}_{2} ) ,$$

wherein S₁ denotes the free oil content in the core sample, C₁ denotes the accumulated amount of light hydrocarbon loss, and C₂ denotes the total accumulated amount of pyrolyzed light hydrocarbons.

Specifically, the relationship among the free oil content S₁, the accumulated amount of light hydrocarbon loss C₁, and the total accumulated amount of pyrolyzed light hydrocarbons C₂ is illustrated as follows. The free oil content S₁ indicates a remaining hydrocarbon amount after C₁ was lost when the core sample reaches the pyrolysis tank (corresponding to the core sample reaching a storage point in actual practice). Therefore, S₁+C₁ denotes an original total amount of hydrocarbons in the core sample. Concerning C₂, the total amount of pyrolyzed light hydrocarbon obtained through pyrolysis in this embodiment corresponds to an amount of light hydrocarbons further volatilized and thus lost during the actual storage of the core sample (which is achieved by controlling a pyrolysis temperature parameter, in which case only some of the hydrocarbons will be pyrolyzed, corresponding to the volatilization and loss in the actual storage of the core sample). Therefore, S₁−C₂ denotes a remaining hydrocarbon amount in the core sample with C₂ being further lost after storage in the actual exploration. In exploration and analysis, the actual hydrocarbon amount in the core sample is S₁+C₁, while the data obtained through pyrolysis is S₁−C₂, a quotient of which denotes the light hydrocarbon correction coefficient w.

With the method proposed in this embodiment, the light hydrocarbon correction coefficient w for the core sample is determined. In the subsequent actual exploration, for the core sample from the same location and with similar lithofacies, the original and real data of the hydrocarbon content in the target formation corresponding to said core sample, i.e., the oil content information, can be directly obtained based on measured data combined with the corresponding light hydrocarbon correction coefficient w. In this manner, the amount of hydrocarbon loss C₁ during the extraction of core sample and the amount of hydrocarbon loss C₂ during the subsequent storage will not affect the accurate evaluation of the hydrocarbon amount in the target formation.

Further, the free oil content can be measured through the following steps:
Step S211a: separating and freezing a sub-sample from the core sample before the pyrolysis of the core sample; and
Step S212a: pyrolyzing the frozen sub-sample, and determining the free oil content based on the amount of pyrolysis product.

Further, the total accumulated amount of pyrolyzed light hydrocarbons is measured through the following steps:
Step S211b: determining the total accumulated amount of hydrocarbon signal when the hydrocarbon signal stops;
Step S212b: generating a calibration curve based on a response to the hydrocarbon signal of the pyrolysis of a standard light oil sample; and
Step S213b: determining the total accumulated amount of pyrolyzed light hydrocarbons of the core sample based on the calibration curve and the total accumulated amount of hydrocarbon signal.

Specifically, the calibration curve needs to be determined in advance through pyrolysis of the standard light oil sample based on the response to the hydrocarbons in the pyrolysis product of the standard light oil sample and a volatile mass of the standard light oil sample. Therefore, for subsequent pyrolysis of the core sample, the amount of pyrolyzed light hydrocarbons can be determined based on the hydrocarbon signal of the pyrolysis product of the core sample by referring to the calibration curve. That is, in Step S100, the amount of pyrolyzed light hydrocarbons at a certain time can be determined based on the hydrocarbon signal amount and the calibration curve, and in Step S212b, the total amount of pyrolyzed light hydrocarbons can be determined based on the total accumulated amount of hydrocarbon signal and the calibration curve.

In addition, when determining the amount of pyrolyzed light hydrocarbons at a certain time based on the amount of hydrocarbon signal and the calibration curve and determining the total amount of pyrolyzed light hydrocarbons based on the total accumulated amount of hydrocarbon signal and the calibration curve, the blank baseline needs to be deducted from the amount of hydrocarbon signal and the total accumulated amount of hydrocarbon signal. The blank baseline is obtained in advance and run for no less than 3600 minutes.

It should be noted that all the data in this embodiment, including the free oil content, the accumulated amount of light hydrocarbon loss, the accumulated amount of pyrolyzed light hydrocarbons, and the total accumulated amount of pyrolyzed light hydrocarbons, all refer to percentages relative to the mass of the core sample.

### Embodiment 3

The embodiment of the present invention proposes a loss recovery and correction method for light hydrocarbons in mud shale. This embodiment mainly illustrates further improvements of the method according to the present invention, wherein duplicate content same as in Example 2 will not be repeated here. The method according to this embodiment includes:
Step S100: determining, based on the hydrocarbon signal in response to the hydrocarbons in the pyrolysis product of the core sample, a relationship between an accumulated amount of light hydrocarbons pyrolyzed from the core sample and the pyrolysis time, and determining, according to said relationship, an accumulated amount of light hydrocarbon loss in the core sample during a time period after extraction from target formation and prior to pyrolysis;
Step S200: determining, according to the accumulated amount of light hydrocarbon loss, a light hydrocarbon correction coefficient, based on which the oil content information of target formation corresponding to other core samples is obtained; and
Step S300: collecting the pyrolysis product of the core sample in different time periods, in order to determine the amount of pyrolyzed hydrocarbons, proportions of hydrocarbon components and the content of each component corresponding to different time periods.

Specifically, in addition to quantitative detection based on the hydrocarbon signal responding to the core sample, qualitative detection can be further carried out. Different hydrocarbon components in the pyrolysis product at different times as well as the content and proportion of each component can be obtained. In this manner, the specific types of light hydrocarbons lost from the core sample in the early stage and the amount thereof can be determined reversely, providing a basis for subsequent research and analysis. The measured results of the proportions of different hydrocarbon components in the pyrolysis product at different times are shown in FIG. 7.

In addition, in actual application, two parallel detection and analysis terminals (e.g., the first detection unit and the second detection unit in Embodiment 1) are provided for the response to the hydrocarbon signal of the pyrolysis product of the core sample and the component analysis of the pyrolysis product thereof, for quantitative and qualitative analysis respectively. Reference can be made to the apparatus as shown in FIG. 1. At this time, the pyrolysis product of the core sample is actually separated and enters the two detection and analysis terminals respectively. Therefore, the accumulated amount of pyrolyzed light hydrocarbons and the total accumulated amount of pyrolyzed light hydrocarbons determined by the method in Embodiment 2 only take into account the hydrocarbon reaching the detection and analysis terminals, rather than all the original hydrocarbons in the core sample. Therefore, it is necessary to further determine the real data of the core sample according to the flow distribution ratio of the two detection and analysis terminals.

### Embodiment 4

The embodiment of the present invention proposes a loss recovery and correction method for light hydrocarbons in mud shale. This embodiment further illustrates the method according to the present invention based on Embodiment 2 in combination with actual data in exploration. At the drilling site, nine fresh Jurassic samples from Well A in Sichuan Basin (China) are selected. The samples include grayish-black mudstone, grayish-black silty mudstone and black shale, etc. See the following table for detailed information.

TABLE 1
Typical Jurassic Samples from Well A in Sichuan Basin

| | | Sample | | | Sample | Free Oil |
|---|---|---|---|---|---|---|
| Serial | | Depth | | Median | Weight | Content |
| Number | Sample | (m) | Lithology | Time | (g) | S₁ (mg/g) |
| 1 | LY1-1-E | 2745.73 | Grayish-black | ****/2/12 | 1173 | 7.71 |
| | | | mudstone | 3:31:30 | | |
| 2 | LY1-2-E | 2760.66 | Grayish-black | ****/2/14 | 1350 | 0.13 |
| | | | silty mudstone | 15:19:00 | | |
| 3 | LY1-3-E | 2783.1 | Blackish-gray | ****/2/17 | 697 | 2.01 |
| | | | silty shale | 22:08:00 | | |
| 4 | LY1-7-E | 2906.99 | Black shale | ****/3/2 | 580 | 4.86 |
| | | | | 20:10:00 | | |
| 5 | LY1-8-E | 2922.31 | Black shale | ****/3/4 | 664 | 3.69 |
| | | | interbedded with | 20:52:30 | | |
| | | | limestone bands | | | |
| 6 | LY1-9-E | 2938.27 | Black shale | ****/3/8 | 585 | 7.72 |
| | | | | 5:02:30 | | |
| 7 | LY1-10-E | 2947.12 | Black shale | ****/3/10 | 832 | 6.86 |
| | | | | 5:45:00 | | |
| 8 | LY1-11-E | 3038.33 | Black shale | ****/3/15 | 711 | 3.75 |
| | | | interbedded with | 0:47:30 | | |
| | | | shell laminae | | | |
| 9 | LY1-12-E | 3047.79 | Black shale | ****/3/17 | 588 | 2 |
| | | | interbedded with | 12:00:00 | | |
| | | | shell bands | | | |

Referring to Embodiment 2, the method according to this embodiment includes the following steps.

Step S100: determining, based on the hydrocarbon signal in response to the hydrocarbons in the pyrolysis product of the core sample, a relationship between an accumulated amount of light hydrocarbons pyrolyzed from the core sample and the pyrolysis time, and determining, according to said relationship, an accumulated amount of light hydrocarbon loss in the core sample during a time period after extraction from target formation and prior to pyrolysis.

Specifically, after the core is removed from the core barrel, samples with typical lithologies and different oil-content levels are selected based on on-site observation on the core. A time when the core barrel is lifted and a time when it arrives at the wellhead are recorded respectively. A median of the above two time values is calculated as the median time through tripping and the starting time of the loss. A full-diameter core sample with a thickness of 5-6 cm is collected on site, with mud on the surface thereof removed. Then a sub-sample is taken and the free oil content thereof is determined. That is, part of the sample (about 5 grams) is knocked off from the core sample to carry out on-site pyrolysis on the frozen crushed rock sample, so as to obtain the free oil content S₁ content (see Table 1). With the full-diameter core intact, the remaining sample is weighed to record the mass thereof. The sample is placed into the pyrolysis apparatus to carry out core pyrolysis experiment.

According to the preset heating scheme, a heating program is set at an initial mud temperature (about 60° C.), which is kept for about 180 minutes. Then the temperature is increased to 150° C. at a constant rate of 1° C./min. A pyrolysis curve of hydrogen flame ionization detector FID (responding to the hydrocarbons in the pyrolysis product of the core sample) during the pyrolysis is observed. When the pyrolysis curve falls back to the baseline before operation, the pyrolysis of the core sample is completed. FIG. 5 is a diagram showing pyrolysis results of a core sample with a burial depth of 4303.42 meters pyrolyzed by the above preset heating scheme, wherein the abscissa denotes pyrolysis time, and the ordinate denotes an amount of pyrolyzed light hydrocarbons at the corresponding time.

FIG. 6 shows a scatter diagram and its fitting result of an accumulated amount of pyrolyzed light hydrocarbons and a square root of the pyrolysis time (t^{0.5}) of the core sample with the burial depth of 4303.42 meters based on the pyrolysis results as shown in FIG. 5. In FIG. 6, data on light hydrocarbon loss obtained based on the fitting result of the data in the first three hours (180 minutes) after the start of pyrolysis is shown.

The method according to this embodiment further includes the following steps.
Step S110: determining, according to the hydrocarbon signal, a scatter diagram of the accumulated amount of pyrolyzed light hydrocarbons and the pyrolysis time within a time period after the pyrolysis starts;
Step S120: fitting a target curve representing said relationship according to the scatter diagram;
Step S130: regressing the target curve to an initial time point when the pyrolysis starts, and taking the accumulated amount of pyrolyzed light hydrocarbons corresponding to the initial time point as the accumulated amount of light hydrocarbon loss;
Step S200: determining, according to the accumulated amount of light hydrocarbon loss, a light hydrocarbon correction coefficient, based on which the oil content information of target formation corresponding to other core samples is obtained; and
Step S210: determining the light hydrocarbon correction coefficient according to the accumulated amount of light hydrocarbon loss, the total accumulated amount of pyrolyzed light hydrocarbons during the pyrolysis of the core sample, and synchronously-measured free oil content in the core sample. The light hydrocarbon correction coefficient can be determined with an expression as follows:

$$w = ( {S}_{1} + {C}_{1} ) / ( {S}_{1} - {C}_{2} ) ,$$

wherein S₁ denotes the free oil content in the core sample, C₁ denotes the accumulated amount of light hydrocarbon loss, and C₂ denotes the total accumulated amount of pyrolyzed light hydrocarbons.

Specifically, the relationship among the free oil content S₁, the accumulated amount of light hydrocarbon loss C₁, and the total accumulated amount of pyrolyzed light hydrocarbons C₂ is illustrated as follows. The free oil content S₁ indicates a remaining hydrocarbon amount after C₁ was lost when the core sample reaches the pyrolysis tank (corresponding to the core sample reaching a storage point in actual practice). Therefore, S₁+C₁ denotes an original total amount of hydrocarbons in the core sample. Concerning C₂, the total amount of pyrolyzed light hydrocarbon obtained through pyrolysis in this embodiment corresponds to an amount of light hydrocarbons further volatilized and thus lost during the actual storage of the core sample. Therefore, S₁−C₂ denotes a remaining hydrocarbon amount in the core sample with C₂ being further lost after storage in the actual exploration. In exploration and analysis, the actual hydrocarbon amount in the core sample is S₁+C₁, while the data obtained through pyrolysis is S₁−C₂, a quotient of which denotes the light hydrocarbon correction coefficient w.

With the method proposed in this embodiment, the light hydrocarbon correction coefficient w for the core sample is determined. In the subsequent actual exploration, for the core sample from the same location and with similar lithofacies, the original and real data of the hydrocarbon content in the target formation corresponding to said core sample, i.e., the oil content information, can be directly obtained based on measured data combined with the corresponding light hydrocarbon correction coefficient w.

Further, the free oil content can be measured through the following steps:
Step S211a: separating and freezing a sub-sample from the core sample before the pyrolysis of the core sample; and
Step S212a: pyrolyzing the frozen sub-sample, and determining the free oil content based on the amount of pyrolysis product.

Further, the total accumulated amount of pyrolyzed light hydrocarbons is measured through the following steps:
Step S211b: determining the total accumulated amount of hydrocarbon signal when the hydrocarbon signal stops;
Step S212b: generating a calibration curve based on a response to the hydrocarbon signal of the pyrolysis of a standard light oil sample; and
Step S213b: determining the total accumulated amount of pyrolyzed light hydrocarbons of the core sample based on the calibration curve and the total accumulated amount of hydrocarbon signal.

Specifically, a calibration curve (as shown in FIG. 3) is created in advance based on a relationship between calibrated hydrocarbon signal response and oil content for existing crude oil sample in this area, and a blank running baseline of the detection unit for more than 3600 minutes is obtained.

After the above pyrolysis procedure, the amount of light hydrocarbon loss (C₁) and the total accumulated amount of pyrolyzed light hydrocarbons (C₂) of each sample during tripping are calculated according to the pyrolysis data, thus calculating the recovery correction coefficient w of the sample. The results are shown in Table 2 below.

TABLE 2
Test results on light hydrocarbons of typical
Jurassic samples from Well A in Sichuan Basin

| | | | | | | | Light |
|---|---|---|---|---|---|---|---|
| | | | | Free Oil | | | Hydrocarbon |
| | | | Sample | Content | | | Correction |
| Serial | | Sample | Weight | S₁ | C₁ | C₂ | Coefficient |
| Number | Sample | Depth | (g) | (mg/g) | (mg/g) | (mg/g) | w |
| 1 | LY1-1-E | 2745.73 | 1173 | 7.71 | 0.11 | 2.85 | 1.61 |
| 2 | LY1-2-E | 2760.66 | 1350 | 0.13 | 0.00 | 0.04 | 1.47 |
| 3 | LY1-3-E | 2783.1 | 697 | 2.01 | 0.00 | 0.71 | 1.55 |
| 4 | LY1-7-E | 2906.99 | 580.0 | 4.86 | 0.01 | 1.87 | 1.63 |
| 5 | LY1-8-E | 2922.31 | 664.0 | 3.69 | 0.82 | 1.88 | 2.49 |
| 6 | LY1-9-E | 2938.27 | 585.0 | 7.72 | 1.07 | 3.29 | 1.99 |
| 7 | LY1-10-E | 2947.12 | 832.0 | 6.86 | 0.78 | 3.11 | 2.04 |
| 8 | LY1-11-E | 3038.33 | 711.0 | 3.75 | 0.91 | 2.04 | 2.73 |
| 9 | LY1-12-E | 3047.79 | 588 | 2 | 0.54 | 0.99 | 2.51 |

According to the test results of the core samples, it can be determined that the maturity of the core samples and the light hydrocarbon recovery correction coefficient generally increases along with the increase in burial depth. The data of the core samples 1-3 in this embodiment are the test results of representative lithofacies 1 from the same layer, an average recovery correction coefficient of which is 1.54. Therefore, a recovery correction coefficient of lithofacies 1 from the same layer in adjacent wells in this area can be also calculated as 1.54. Similarly, a recovery coefficient of representative lithofacies from other layers can be calculated as 2.04 and 2.62 respectively.

Based on this recovery correction coefficient, there is no need to worry about the influence of light hydrocarbon volatilized loss of the core sample during the extraction and the storage on the analysis results in subsequent pyrolysis of corresponding lithofacies from the same layer. The actual oil content information of the corresponding layer can be determined only based on the pyrolysis results combined with the recovery correction coefficient (a product of the amount of pyrolyzed hydrocarbons and the recovery correction coefficient).

### Embodiment 5

The embodiment in the present invention proposes a pyrolysis system for light hydrocarbons in a core of mud shale, which comprises said pyrolysis apparatus for loss recovery and correction of light hydrocarbons in mud shale, and a data processing apparatus electrically connected thereto. The pyrolysis system can achieve all the technical effects of the pyrolysis apparatus.

In the present invention, it should be understood that the terms “upper”, “lower”, “bottom”, “top”, “front”, “rear”, “internal”, “external”, “left”, “right” and the like indicate orientations or positions based on those shown in the drawings, which are used only for simplified and illustrative purposes of the present invention, and are not intended to indicate or imply a particular orientation, or the configuration and operation of a device or element in a particular orientation. Therefore, the above terms are not intended to restrict the present invention.

Although the present invention is described hereinabove with reference to the particular embodiments, it should be understood that these embodiments are provided to illustrate the principle and the application of the present invention merely. Therefore, it is possible to modify the exemplary embodiments and define other arrangements as long as they fall within the spirit and scope of the present invention defined in the appending claims. Different dependent claims and the technical features described in this context may be combined in a manner different from those in the original claims. It is also to be understood that the technical features described in combination with separate embodiments may be applied to other embodiments as described.

### LIST OF REFERENCE SIGNS

1 carrier gas assembly; 2 pyrolysis tank: 21 input pipeline; 22 output pipeline; 221 first branch pipeline; 222 second branch pipeline; 223 third branch pipeline; 23 temperature sensor; 24 heating unit; 25 tank body; 251 vertical air grille structure; 26 quick-release structure; 261 cover; 262 core basket; 3 flow divider assembly; 31 triple valve; 32 flow divider valve; 4 detection assembly; 41 first detection unit; 42 second detection unit; 5 combustion mechanism; 51 combustion gas unit; 52 combustion supporting gas unit; 6 thermal insulation box; 7 flow controller; and 8 core sample.

1. A pyrolysis apparatus for loss recovery and correction of light hydrocarbons in mud shale, comprising:
a pyrolysis tank, for accommodating and heating a full-diameter core sample, an input pipeline and an output pipeline being respectively arranged at two opposite ends of the pyrolysis tank, and a flow divider assembly being arranged on the output pipeline, forming a first branch pipeline and a second branch pipeline;
a carrier gas assembly in communication with the input pipeline; and
a detection assembly, comprising a first detection unit for quantitative detection and a second detection unit for qualitative detection, which are connected to the first branch pipeline and the second branch pipeline, respectively,
wherein the pyrolysis tank comprises an oval columnar tank body, a heating mechanism is arranged on a side wall of the tank body, and a vertical air grille structure is arranged on each of front and rear sides of the tank body, the vertical air grille structure including several gas holes vertically arranged in a row.

2. The pyrolysis apparatus according to claim 1, characterized in that the flow divider assembly comprises a triple valve and a flow divider valve, wherein the triple valve is configured to separate the output pipeline to form the first branch pipeline and the second branch pipeline, and the flow divider valve is arranged on the first branch pipeline or the second branch pipeline, for controlling a flow distribution proportion of the first branch pipeline to the second branch pipeline, the flow divider valve being configured as a high-temperature resistant metal flow divider valve.

3. The pyrolysis apparatus according to claim 1, characterized in that the apparatus further comprises a thermal insulation box arranged at an output end of the pyrolysis tank, wherein the output pipeline is arranged inside the thermal insulation box.

4. The pyrolysis apparatus according to claim 1, characterized in that a third branch pipeline is formed at an end of the second branch pipeline and configured as an exhaust pipeline with an exhaust valve, and
the first branch pipeline and the second branch pipeline are in a normally open state under a normal pressure.

5. The pyrolysis apparatus according to claim 1, characterized in that temperature sensors are arranged on the pyrolysis tank at least in a position where the heating mechanism is located and at an output end thereof corresponding to the output pipeline; and
the tank body is of a multi-layer structure, the heating mechanism is configured as an annular heating sheet arranged as a middle layer of the tank body, and a quick-release structure is arranged on the tank body and comprises a cover arranged on a top portion of the tank body and connected to a core basket through a vertical connecting member.

6. A loss recovery and correction method for light hydrocarbons in mud shale, comprising:
determining, based on hydrocarbon signal in response to hydrocarbons in a pyrolysis product of a core sample, a relationship between an accumulated amount of pyrolyzed light hydrocarbons of the core sample and a pyrolysis time;
determining, according to said relationship, an accumulated amount of light hydrocarbon loss of the core sample during a time period after extraction from target formation and prior to pyrolysis; and
determining, according to the accumulated amount of light hydrocarbon loss, a light hydrocarbon correction coefficient.

7. The method according to claim 6, characterized in that the hydrocarbon signal of the hydrocarbons in the pyrolysis product of the core sample is detected by the pyrolysis apparatus.

8. The method according to claim 6, characterized in that determining, according to said relationship, the accumulated amount of light hydrocarbon loss of the core sample during the time period after extraction from target formation and prior to pyrolysis comprises:
determining, according to the hydrocarbon signal, a scatter diagram of the accumulated amount of pyrolyzed light hydrocarbons and the pyrolysis time within a time period after the pyrolysis starts;
fitting a target curve representing said relationship according to the scatter diagram; and
regressing the target curve to an initial time point when the pyrolysis starts, and taking the accumulated amount of pyrolyzed light hydrocarbons corresponding to the initial time point as the accumulated amount of light hydrocarbon loss.

9. The method according to claim 8, characterized in that determining, according to the accumulated amount of light hydrocarbon loss, the light hydrocarbon correction coefficient comprises:
determining the light hydrocarbon correction coefficient according to the accumulated amount of light hydrocarbon loss, a total accumulated amount of pyrolyzed light hydrocarbons during the pyrolysis of the core sample, and synchronously-measured free oil content in the core sample.

10. The method according to claim 9, characterized in that the light hydrocarbon correction coefficient is determined with an expression as follows:
$$w = ( {S}_{1} + {C}_{1} ) / ( {S}_{1} - {C}_{2} ) ,$$
wherein S₁ denotes the free oil content in the core sample, C₁ denotes the accumulated amount of light hydrocarbon loss, and C₂ denotes the total accumulated amount of pyrolyzed light hydrocarbons.

11. The method according to claim 9, characterized in that the free oil content is measured through steps of:
separating and freezing a sub-sample from the core sample before the pyrolysis of the core sample; and
pyrolyzing the frozen sub-sample and determining the free oil content based on an amount of pyrolysis product of the sub-sample.

12. The method according to claim 9, characterized in that the total accumulated amount of pyrolyzed light hydrocarbons is measured through steps of:
determining a total accumulated amount of hydrocarbon signal when the hydrocarbon signal stops; and
determining the total accumulated amount of pyrolyzed light hydrocarbons of the core sample according to the total accumulated amount of hydrocarbon signal.

13. The method according to claim 12, characterized in that determining the total accumulated amount of pyrolyzed light hydrocarbons of the core sample according to the total accumulated amount of hydrocarbon signal comprises:
generating a calibration curve based on a response to the hydrocarbon signal of the pyrolysis of a standard light oil sample; and
determining the total accumulated amount of pyrolyzed light hydrocarbons of the core sample based on the calibration curve and the total accumulated amount of hydrocarbon signal.

14. The method according to claim 6, characterized in that the method further comprises:
collecting the pyrolysis product of the core sample in different time periods, in order to determine the amount of pyrolyzed hydrocarbons, proportions of hydrocarbon components and the content of each component corresponding to said different time periods.

15. A pyrolysis system for loss recovery and correction of light hydrocarbons in mud shale, comprising said pyrolysis apparatus according to claim 1, and a data processing apparatus electrically connected thereto.
